(19) United States
(12) Patent Application Publication
(10) Pub. No.: US 2024/0384064 A1
(43) Pub. Date: Nov. 21, 2024

(54) 3D PRINTING RESIN WITH SEPARATION EFFECT

(71) Applicant: PRO3DURE MEDICAL GMBH, Dortmund (DE)

(72) Inventor: Martin KLARE, Dortmund (DE)

(73) Assignee: PRO3DURE MEDICAL GMBH, Dortmund (DE)

(21) Appl. No.: 18/693,961

(22) PCT Filed: Sep. 22, 2022
(86) PCT No.: PCT/EP2022/076450

§ 371 Date: Mar. 21, 2024

(30) Foreign Application Priority Data
Sep. 22, 2021 (EP) 21198394.5

(51) Int. Cl. C08K 5/101; B33Y 70/00; C08K 5/3475; C08K 5/45

(52) U.S. Cl. CPC C08K 5/101; C08K 5/3475; C08K 5/45; B33Y 70/00

(57) ABSTRACT

The present invention relates to a 3D printing resin comprising a monomer or oligomer having at least one acrylate or methacrylate subunit and an initiator. The 3D printing resin is characterized in that it comprises a saturated fatty acid or a carboxylic acid ester, whereby a separation effect is achieved with respect to other plastics. The invention further relates to a polymer comprising the monomers and oligomers described above in polymerized form and a saturated fatty acid or carboxylic acid ester. If only 60-80% of the double bonds of the monomer or oligomer are reacted, a green compact can be obtained. The invention also provides a kit-of-parts system comprising the above-described 3D printing resin for producing a polymer with a separation effect and a powder component and a liquid component for producing a plastic. Finally, the invention also relates to methods for producing a molded part with a separation effect and to a method for producing a molded part, wherein the molded part is complementary to another molded part. In the latter method, one molded part is made from the 3D printing resin described above and the other molded part is made from a plastic.

The present invention relates to a 3D printing resin with a separation effect as well as molded parts produced therefrom and their manufacturing process.

### BACKGROUND TO THE INVENTION

The aim of the present invention is to provide a 3D printing resin with an inherent separation effect with respect to other plastic systems. 3D printed objects made of the material according to the invention can be advantageously used in the medical technology sector and in particular in the dental sector in combination with traditional manufacturing methods. An example of this is the manufacture of orthodontic appliances using the so-called “salt and pepper” technique. A powder-liquid system is gradually applied to a dental model by sprinkling small amounts of powder (preferably polymethyl methacrylate polymer powder) with monomeric methyl methacrylate until a paste-like consistency is achieved. The powder/liquid system is mixed with an initiator system so that in rare cases direct autopolymerization takes place on the dental model or in most cases the model with the pasty, sprinkled object is placed in a pressure pot and cured under pressure and heat. Traditionally, dental models made of plaster are used for this, which are sprayed with an insulator solution before the scattering plastic is applied to them. After curing, the objects from the powder/liquid system can then be easily separated from the plaster model and finished (e.g. ground and polished).

Nowadays, however, there is a shift to “digital workflows” based on biometric data. This means that a 3D-printed dental model based on scan data forms the working basis for the dental technician. Such models are no longer made of plaster, but are produced from synthetic resins in a layering process (e.g. using an image projection system such as the ASIGA MAX UV from Asiga, Sydney). This results in several problems. On the one hand, the monomer component of the powder-liquid system can dissolve the polymer network of the dental model plastic, penetrate the network and form an “interpenetrating network”. After curing, there is then a bond with the dental model and the object can no longer be detached from the model without damage. On the other hand, such 3D-printed models consist of layers and therefore have inhomogeneous rather than smooth surfaces. This can result in additional mechanical retention, which is intensified by the “tearing of the insulator film” at e.g. undercuts or flat slopes of the model. In addition, the wetting of such polymer surfaces is only complete if an insulator system optimized for the surface properties of the plastic is used. Accordingly, a.o. gels with separation properties (e.g. glycerine-based gels) are used today. The manual application of such gels is time-consuming and always inhomogeneous, especially on undercuts and in interdental spaces. In addition, it is extremely tedious to ensure that the working base is completely coated. As a result, it is not always possible to avoid a bond between the dental model and the scattering plastic after curing. In the worst-case scenario, parts of the appliance break off during removal and the process has to be completely repeated. Another important disadvantage of this procedure is that different layer thicknesses and inhomogeneities when applying the separator gel result in a surface for the scattering plastic that no longer accurately represents the anatomical conditions. This can result in fitting problems, for example, which may require the orthodontic appliance to be reworked or even remade.

In order to counter all the above-mentioned disadvantages, it is therefore desirable to provide a modeling plastic that has an inherent separation effect, which leads to a homogeneous surface of the printed objects that is insulated from scattering plastics. The 3D printing resin “e-Sepfree” from Envisiontec, Gladbeck, is state of the art for this purpose. Wax particles are added to the material as a filler, which leads to reduced adhesion of scattering plastics to objects built from this resin. However, this special property of the wax particles compared to liquid monomers/polymers also means that the particles separate in a liquid 3D printing resin and the wax particles sink to the bottom of the resin. This can result in faulty build jobs or components with inhomogeneous surface properties. Furthermore, the resin must be mixed regularly to avoid the above-mentioned sources of error. In addition, 3D printing resins filled with wax particles are opaque. Depending on the 3D printing technology selected (e.g. stereolithography with irradiation source from above and sinking of the component into the resin), no build process control can be carried out during the construction process. Accordingly, faulty build processes cannot be aborted in time or modified during the build (e.g. by deleting a component) and thus “saved”.

Other disadvantages include the fact that the wax particles increase the viscosity of the construction resin and reduce the mechanical properties.

The present invention provides a solution to the above-mentioned problems and limitations. It was surprisingly found that 3D printing resins containing >5 m % of a saturated fatty acid or its esters with a chain length<20 C can be used to produce additively manufactured objects that exhibit a significantly increased inherent separation effect compared to the prior art. The claimed resin formulations are homogeneous. In particular, the resins according to the invention do not exhibit any separation or segregation effects. In addition, the invention can be used to provide resin systems which are transparent and which turn opaque white after polymerization. As a result, the generated objects can be optimally observed in the resin vat during the construction job, thus ensuring optimum process control. Compared to systems filled with wax particles, the viscosity of the resins according to the invention is significantly lower. This is advantageous for 3D printing processes for many reasons. On the one hand, the resin flows more easily (depending on the technology) after the construction of each layer and wets the component more quickly. This allows higher construction speeds to be achieved. On the other hand, additively manufactured components can be cleaned more easily and efficiently if the viscosity is lower.

### SUMMARY OF THE INVENTION

A first aspect of the invention relates to a 3D printing resin comprising
a monomer or oligomer which has at least one acrylate or methacrylate subunit,
an initiator,
   characterized in that the 3D printing resin comprises a saturated fatty acid or a carboxylic acid ester.

A second aspect of the invention relates to a polymer comprising polymerized monomers and/or oligomers according to the first aspect, and a saturated fatty acid or carboxylic acid ester.

A third aspect of the invention relates to a green compact comprising polymerized monomers and/or oligomers according to the first aspect, wherein 60% to 80% of the double bonds of the acrylate and/or methacrylate subunits have been reacted, and a saturated fatty acid or a carboxylic acid ester.

A fourth aspect of the invention relates to kit-of-parts system comprising
a 3D printing resin according to the first aspect for producing a polymer with a separation effect, and
a powder component and a liquid component for producing a plastic, wherein the powder component comprises a polymer powder and the liquid component comprises a monomer.

A fifth aspect of the invention relates to a method of manufacturing a molded part having a separation effect, wherein the 3D printing resin according to the first aspect is used and cured.

A sixth aspect of the invention relates to a method of manufacturing a molded part, wherein
in a first step, the method according to the fifth aspect is carried out to obtain a first molded part, and
in a second step, the first molded part is used to obtain a second molded part which is at least partially complementary to the first molded part, the second molded part being made from a plastic, or
in a first step, a first molded part is produced from a plastic, and
in a second step, the first molded part is used to obtain a second molded part which is at least sectionally complementary to the first molded part, wherein the second molded part is produced using the 3D printing resin according to the first aspect.

### TERMS AND DEFINITIONS

The term alkyl refers to a saturated linear or branched hydrocarbon chain. A C₁₋₄-alkyl refers to a hydrocarbon chain comprising 1, 2, 3 or 4 carbon atoms. Examples of C₁₋₄-alkyls are methyl, ethyl, propyl, isopropyl, n-butyl, 2-methylpropyl and tert-butyl.

The term alkenyl refers to a linear or branched hydrocarbon chain that has at least one carbon-carbon double bond in addition to carbon-carbon single bonds.

The term cycloalkyl or cycloaliphatic refers to a saturated monocyclic or polycyclic hydrocarbon compound. Monocyclic hydrocarbon compounds form a ring structure, e.g. cyclohexyl (—C₆H₁₁). Polycyclic hydrocarbon compounds include hydrocarbon compounds that form several rings, e.g. isobornyl or tricyclodecyl. A C₃₋₁₈-cycloalkyl refers to a mono- or polycyclic hydrocarbon compound comprising 3 to 18 carbon atoms. Cycloalkyls may be substituted with one or more C₁₋₄-alkyls, e.g. tricyclodecanedimethanol.

The term heterocyclic refers to a monocyclic or polycyclic hydrocarbon compound in which at least one carbon atom is replaced by a heteroatom, in particular N, O or S. In particular, carbon atoms are only connected to each other via single bonds (saturated heterocyclic compound) or via single bonds and double bonds.

The term initiator refers to molecules that form a radical and thus start a polymerization reaction. The formation of radicals is catalyzed by so-called activators.

The term activator refers to chemical compounds, an increase in temperature, light or high-energy radiation that stimulate an initiator to form radicals. For example, peroxide compounds can be thermally or photochemically stimulated to form radicals.

The term interpenetrating network refers to a polymer network comprising two or more networks that are at least partially cross-linked at the molecular level but are not covalently bonded to each other. The networks cannot be separated unless chemical bonds are broken.

The term dimer fatty acid, dimerized fatty acid or dimer acid refers to a mixture produced by oligomerization, in particular dimerization, of fatty acids (cooking). Oligomerization requires at least one fatty acid with conjugated double bonds as well as other unsaturated fatty acids. In particular, unsaturated C₁₂₋₂₂ fatty acids are used. The reaction is carried out using Diels-Alderaddition, which usually results in the formation of a partially unsaturated C₆ ring. Depending on the number and position of the double bonds of the fatty acids used to produce the dimer fatty acids, mixtures of predominantly dimeric products are formed. If C₁₂₋₂₂ fatty acids are used, the dimers then have 24 to 44 carbon atoms, for example. In particular, fatty acids with 18 carbon atoms are used for production, so that the dimeric product has 36 carbon atoms. In addition to the dimer, trimers and monomers of the fatty acids can also be present in the mixture. Commercially available dimer fatty acids generally contain at least 80% by weight of dimer molecules, up to 19% by weight of trimer molecules and a maximum of 1% by weight of monomer molecules and other by-products. In particular, dimer fatty acids are used which consist of at least 90% by weight, in particular at least 95% by weight, further in particular at least 98% by weight of dimeric fatty acid molecules. Suitable dimer fatty acids with 36 carbon atoms are contained, for example, in the mixture available under “Empol 1062” from the company Cognis.

The term Tinuvin P refers to CAS 2440-22-4.

The term Tinopal OB refers to CAS 7128-64-5.

The term TPO refers to CAS No. 75980-60-8.

The term TPOL refers to CAS No. 84434-11-7.

The term Irgacure 819 refers to CAS No. 162881-26-7.

### DESCRIPTION OF THE INVENTION

A first aspect of the invention relates to a 3D printing resin comprising
a monomer or oligomer which has at least one acrylate or methacrylate subunit,
an initiator,
   characterized in that the 3D printing resin comprises a saturated fatty acid or a carboxylic acid ester.

The 3D printing resin of the present invention can be used to 3D print objects, whereby the initiator is activated and a polymerization reaction takes place. The saturated fatty acids or carboxylic acid esters contained in the 3D printing resin form a film around the polymer structures, whereby a separation effect is achieved with respect to other plastics. As described above, this effect can be helpful in the dental sector, for example, in the production of orthodontic fittings. For example, a replica of the jaw, including the dentition, produced with the help of the 3D printing resin can be used as a positive mold for the production of dental splints. The dental splint can be produced directly on the positive mold using known powder-liquid systems such as a polymerization of polymethyl methacrylate polymer (powder) and methyl methacrylate (liquid component) and can be easily detached from the mold due to the separation effect.

The 3D printing resin can have one or more types of monomers and/or oligomers. A combination of several saturated fatty acids and/or carboxylic acid esters is also possible.

In one embodiment, the fatty acid is an acid according to formula (1),

R¹⁰—COOH (1),

wherein R¹⁰ is a C₁₋₁₉-alkyl, in particular a C₃₋₁₉-alkyl, further in particular a C₁₁₋₁₇-alkyl.

In one embodiment, the carboxylic acid ester is selected from an ester according to formula (2), an ester according to formula (3) or an ester prepared from a dimer fatty acid and two alcohols, in particular C₁₋₁₂-alkyl-OH

R¹¹—C(═O)—O—R¹² (monocarboxylic acid ester) (2),

R¹³—O—C(═O)—R¹⁴—C(═O)—O—R¹⁵ (dicarboxylic acid ester) (3),
whereby
R¹¹ is a C₁₋₂₃-alkyl or a C₂₋₂₃-alkenyl, in particular a C₃₋₂₃-alkyl or a C₃₋₂₃-alkenyl, wherein the alkyl or the alkenyl is unsubstituted or substituted by one or more, in particular one, —OH,
R¹², R¹³ and R¹⁵ is a C₁₋₁₂-alkyl,
R¹⁴ is a C₁₋₁₂-alkyl or a C₂₋₁₂-alkenyl, in particular a C₂₋₁₂-alkyl or a C₂₋₁₂-alkenyl.

The carboxylic acid ester can contain a saturated or unsaturated acid portion. As a rule, the acid portion is unsubstituted. However, slightly substituted esters such as a ricinoleic acid ester are also suitable.

In one embodiment, R¹¹ is unsubstituted.

In one embodiment, the dimer fatty acid is an acid according to formula (5),

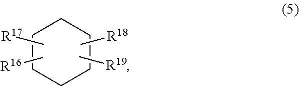

whereby
R¹⁶ and R¹⁷ are independently of one another a C₁₋₂₃-alkyl or a C₂₋₂₃-alkenyl, in particular a C₃₋₂₃-alkyl or a C₃₋₂₃-alkenyl, the alkyl or the alkenyl being unsubstituted or substituted by one or more, in particular one, —OH,
R¹⁸ and R¹⁹ are independently of one another C₁₋₂₃-alkyl-COOH or C₂₋₂₃-alkenyl-COOH, in particular C₃₋₂₃-alkyl-COOH or C₃₋₂₃-alkenyl-COOH, the alkyl or the alkenyl being unsubstituted or substituted by one or more, in particular one, —OH.

In one embodiment, R¹⁶, R¹⁷, R¹⁸ and R¹⁹ are unsubstituted.

In one embodiment, the sum of all C atoms of the carboxylic acid ester is ≤20, in particular 4-20, more particularly 12-18.

In one embodiment, the proportion by weight of the fatty acid or carboxylic acid ester relative to the total mass of the 3D printing resin is ≥5%.

In one embodiment, the acid component of the carboxylic acid ester is selected from:

n-Butanoic acid (butyric acid), 2-methylpropanoic acid (isobutyric acid), pentanoic acid (valeric acid), i-pentanoic acid, such as 2,2-dimethylpropanoic acid (pivalic acid, neopentanoic acid) and 3-methylbutanoic acid (iso-pentanoic acid, iso-valeric acid), hexanoic acid (caproic acid), heptanoic acid, octanoic acid (caprylic acid), i-octanoic acid such as e.g. in particular 2-propylheptyl-2-ethylhexanoic acid, but also 2-propylheptyl-3-ethylhexanoic acid, 2-propylheptyl-4-ethylhexanoic acid, 2-propylheptyl-5-ethylhexanoic acid and technical mixtures of branched octanoic acids, such as those marketed by Exxon under the trade name Cekanoic® C8. Nonanoic acid (pelargonic acid, nonilic acid), decanoic acid (capric acid), i-decanoic acids, such as trimethylheptanoic acid (neodecanoic acid, isodecanoic acid) and technical mixtures of branched decanoic acids, such as those sold under the trade name Cekanoic® C10 by the company Exxon, undecanoic acids and technical mixtures of branched decanoic acids, such as those sold under the trade name Cekanoic® C10 by the company Exxon. Exxon, undecanoic acid, undecenoic acid, dodecanoic acid (lauric acid), tridecanoic acid, tetradecanoic acid (myristic acid), pentadecanoic acid, hexadecanoic acid (palmitic acid), heptadecanoic acid (margaric acid), Octadecanoic acid (stearic acid), nonadecanoic acid, eicosanoic acid, docosanoic acid, tetracosanoic acid, hexacosanoic acid, dimer fatty acids (C36, such as those available under the trade name “Empol 1062” from the company Cognis), talc fatty acids, coconut fatty acids, palm fatty acids, ricinoleic acid, oleic acid, linoleic acid, linolenic acid, isostearic acid, isooctanoic acid, isononanoic acid, isodecanoic acid, 2-ethylhexanoic acid, 2-propylheptanoic acid, 2-butyloctanoic acid, 2-butyldecanoic acid, 2-hexyloctanoic acid, 2-hexyldecanoic acid, 2-hexyldodecanoic acid, 2-octyldecanoic acid, fumaric acid, maleic acid, adipic acid, pimelic acid, cork acid, azelaic acid, sebacic acid. Also suitable are esters with Cekanoic®C8 (isooctanoic acid), Cekanoic® C9 (isononanoic acid: 3,5,5-trime-thylhexanoic acid and 2,5,5-trimethylhexanoic acid) and Cekanoic® C10 (isodecanoic acid) from Exxon Mobile, which are mixtures of carboxylic acid isomers.

In one embodiment, the alcohol portion of the carboxylic acid ester is selected from methanol, ethanol, propanol, isopropanol, butanol, isobutanol, pentanol, hexanol, isohexanol, octanol, decanol and dodecanol.

In one embodiment, the monomer or oligomer comprises, in addition to the at least one acrylate or methacrylate subunit, one or more subunits selected from an aliphatic, a cycloaliphatic, a heterocyclic, an aromatic and a urethane subunit.

In one embodiment, the monomer is selected from a compound of formula (4),

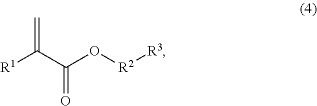

whereby
R¹ is equal to —H or —CH₃,
R² is selected from
   C₁- to C₁₀-alkyl,
   —[(CH₂)ₘ—O—]ₙ—(CH₂)ᵣ—,
   —[(C₁₋₁₂-alkyl)ₛ-Y]ₓ—(CH₂)ₜ,

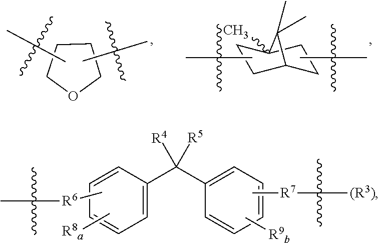

whereby
R⁴ and R⁵ are independently selected from H, —C₁₋₄-alkyl, —CF₃, phenyl, or
R⁴ and R⁵ form a ring structure comprising a hydrocarbon chain comprising 4 to 8 carbon atoms,
R⁶ is selected from —[(CH₂)ₚ—O—]_{q}—
R⁷ is selected from —O—[(CH₂)ᵥ—O—]_{w}—,
R⁸ₐ and R⁹_{b} are independently selected from —C₁₋₄-alkyl, phenyl,
Y is selected from —O—C(═O)—NR²⁰— and —NR²⁰—C(═O)—O—,
R²⁰ is a C₁₋₄-alkyl or H,
m, p, v is between 1 and 4, in particular 1 or 2,
n, q, w is between 1 and 4,
r, t, x is between 1 and 4, in particular 1 or 2,
s is between 1 and 10,
a is 0 or 1,
b is 0 or 1, and
R³ is —H, acrylate or methacrylate.

In one embodiment, the monomer is selected from:
Dimethacrylates of (n)-alkoxylated bisphenol A such as bisphenol A ethoxylate(2) dimethacrylate, bisphenol A ethoxylate(4) dimethacrylate, bisphenol A propoxylate(2) dimethacrylate, bisphenol A propoxylate(4) dimethacrylate and dimethacrylates of (n)-alkoxylated bisphenol F such as bisphenol F ethoxylate(2) dimethacrylate and bisphenol F ethoxylate(4) dimethacrylate, bisphenol F-propoxylate(2) dimethacrylate, bisphenol F propoxylate(4) dimethacrylate and mixtures thereof. Preferably, monomeric or oligomeric dimethacrylates based on bisphenol A are used, in particular bisphenol A ethoxylate(2) dimethacrylate and bisphenol A ethoxylate (4)dimethacrylate. Particularly preferred are mixtures of these two dimethacrylates based on bisphenol A, with a proportion of bisphenol A ethoxylate(4) dimethacrylate>bisphenol A ethoxylate(2) dimethacrylate.
Urethane (meth)acrylates with a functionality<4 are known to the skilled person and can be produced in a known manner, for example by reacting a hydroxyl-terminated polyurethane with methacrylic acid to form the corresponding urethane methacrylate, or by reacting an isocyanate-terminated prepolymer with hydroxymethacrylates. Corresponding processes are known, for example, from EP 0579503. Urethane (meth)acrylates are also commercially available and are sold, for example, under the name PC-Cure&reg; by the company Piccadilly Chemicals, under the product name CN 1963 by the company Sartomer, under the name Photomer by the company Cognis, under the name Ebecryl by the company UCB and under the name Genomer&reg; by the company Rahn. Preferred urethane (meth)acrylates are those which are functionalized to n<4, have viscosities<15 Pa s, have a molecular weight<2000 and have been produced from aliphatic reactants. In particular, the isomer mixture 7,7,9-(or 7,9,9-)trimethyl-4,13-dioxo-3,14-dioxa-5,12-diazahexadecane-1,16-diol dimethacrylate obtained from HEMA and TMDI is used.
I,3-butanediol dimethacrylate, 1,6-hexanediol dimethacrylate, 1,3-butylene glycol dimethacrylate, diethylene glycol dimethacrylate, ethylene glycol dimethacrylate, neopentyl dimethacrylate, polyethylene glycol dimethacrylate, triethylene glycol dimethacrylate and tetraethylene glycol dimethacrylate and preferably 1,4-butanediol dimethacrylate. Such products are commercially available, for example from the company Sartomer.
a reaction product of an asymmetrically trimerized, aliphatic isocyanate with a hydroxyl-containing methacrylate, for example 2-hydroxyethyl methacrylate. Such products are commercially available, for example from the company Rahn. The asymmetric variants are characterized by a lower viscosity compared to the symmetric variants.

In one embodiment, the oligomer is formed from the monomers described above.

In one embodiment, the oligomer comprises XX to XX monomers.

When the 3D printing resin is used in a 3D printer, curing/polymerization takes place using UV light. The initiator is split by irradiation with light of a suitable wavelength and a radical is formed, which starts the polymerization of the monomers and/or oligomers.

In one embodiment, the initiator is a photoinitiator.

In one embodiment, the initiator is a photoinitiator selected from
a compound of benzoins and benzoin ethers, in particular benzoin, benzoin methyl ether, benzoin ethyl ether and benzoin isopropyl ether, benzoin phenyl ether and benzoin acetate,
a compound of acetophenones, in particular acetophenone, 2,2-dimethoxyacetophenone, and 1,1-dichloroacetophenone,
a compound of the benzils and benzil ketals, in particular benzil, benzildimethyl ketal and benzildiethyl ketal,
a compound of the anthraquinones, in particular 2-methylanthraquinone, 2-ethylanthraquinone, 2-tert-butylanthraquinone, 1-chloroanthraquinone and 2-amylanthraquinone,
a compound of triphenylphosphines and benzoylphosphine oxides, in particular triphenylphosphines, 2,4,6-trimethylbenzoyldiphenylphosphine oxide (luzirine TPO) and bis(2,4,6-trimethylbenzoylphenyl)-phosphine oxide,
a compound of the benzophenones, in particular benzophenone and 4,4′-bis-(N,N′-dimethylamino)-benzophenone,
a compound of thioxanthones and xanthones, in particular thioxanton and xanthone,
an acridine derivative, phenazine derivative, quinoxaline derivative
1-Phenyl-1,2-propanedione-2-O-benzoyloxime,
a compound of the 1-aminophenylketones or 1-hydroxyphenylketones, in particular 1-hydroxycyclohexylphenylketone, phenyl-(1-hydroxyisopropyl)-ketone and 4-isopropylphenyl-(1-hydroxyisopropyl)-ketone.

In 3D printing processes, additives can be used that either modify the manufactured object, e.g. through colorants, or facilitate the printing process. For example, UV blockers can be used to regulate the penetration depth of UV radiation to start the polymerization reaction or the stability can be influenced by anaerobic or aerobic stabilizers.

In one embodiment, the 3D printing resin comprises one or more additives selected from an inhibitor, a stabilizer, a filler, a colorant, a UV blocker.

A second aspect of the invention relates to a polymer comprising polymerized monomers and/or oligomers according to the first aspect, and a saturated fatty acid or carboxylic acid ester.

The 3D printing resin described above contains monomers and/or oligomers that can form a polymer in a radical polymerization reaction.

In one embodiment, the polymer is made using the 3D printing resin according to the first aspect of the invention.

In one embodiment, the monomers and/or oligomers are fully polymerized.

When fabricating dental models, it can be helpful to first produce a green compact and then cure it completely later.

A third aspect of the invention relates to a green compact comprising polymerized monomers and/or oligomers according to the first aspect, wherein 60% to 80% of the double bonds of the acrylate and/or methacrylate subunits have been reacted, and a saturated fatty acid or a carboxylic acid ester.

In one embodiment, the green compact is manufactured using the 3D printing resin according to the first aspect of the invention.

A fourth aspect of the invention relates to kit-of-parts system comprising
a 3D printing resin according to the first aspect of the invention for producing a polymer with a separation effect, and
a powder component and a liquid component for the production of a plastic, wherein
   the powder component comprises a polymer powder, in particular a polymer powder comprising at least one acrylate or methacrylate subunit, and
   the liquid component comprises a monomer, in particular a monomer comprising at least one acrylate or methacrylate subunit.

In one embodiment, the powder component comprises a polymethyl methacrylate polymer and the liquid component comprises methyl methacrylate.

The 3D printing resin according to the invention is used in particular when two complementary plastic objects, such as a dental model and a precisely fitting dental splint, are to be produced. For example, so that the dental splint can be detached from the dental model, the dental model is produced using the 3D printing resin described above and the dental splint is produced from a known plastic system, such as a powder-liquid system (scattering plastic). The kit-of-parts system can be used for such fabrications. The powder-liquid system usually comprises further components such as an initiator to start the polymerization reaction and possibly other additives (see above). Suitable initiators are known to a person skilled in the art. Due to the separation effect, it is not necessary to use isolator solutions or isolating gels, which enable separation of the complementary objects in known processes.

In one embodiment, the kit-of-parts system does not include insulators.

A fifth aspect of the invention relates to a method of manufacturing a molded part having a separation effect, wherein the 3D printing resin according to the first aspect is used and cured.

In one embodiment, the method according to the fifth aspect uses a 3D printer.

A sixth aspect of the invention relates to a method of manufacturing a molded part, wherein
in a first step, the method according to the fifth aspect is carried out to obtain a first molded part, and
in a second step, the first molded part is used to obtain a second molded part which is at least partially complementary to the first molded part, the second molded part being made from a plastic, in particular using a powder component and a liquid component, wherein
   the powder component comprises a polymer powder, in particular a polymer powder comprising at least one acrylate or methacrylate subunit, and
   the liquid component comprises a monomer, in particular a monomer comprising at least one acrylate or methacrylate subunit, or
in a first step, a first molded part is produced from a plastic, in particular using a powder component and a liquid component, wherein
   the powder component comprises a polymer powder, in particular a polymer powder comprising at least one acrylate or methacrylate subunit, and
   the liquid component comprises a monomer, in particular a monomer comprising at least one acrylate or methacrylate subunit, and
in a second step, the first molded part is used to obtain a second molded part which is at least sectionally complementary to the first molded part, wherein the second molded part is produced using the 3D printing resin according to the first aspect.

In one embodiment
in a first step, the method according to the fifth aspect is carried out to obtain a first molded part, and
in a second step, the first molded part is used to obtain a second molded part which is at least partially complementary to the first molded part, the second molded part being made from a plastic, in particular using a powder component and a liquid component, wherein
   the powder component comprises a polymer powder, in particular a polymer powder comprising at least one acrylate or methacrylate subunit, and
   the liquid component comprises a monomer, in particular a monomer comprising at least one acrylate or methacrylate subunit.

The powder-liquid system usually comprises further components such as an initiator to start the polymerization reaction and possibly other additives (see above). Suitable initiators are known to a specialist. Due to the separation effect, it is not necessary to use isolator solutions or isolating gels, which enable separation of the complementary objects in known processes.

In one embodiment, the method does not include the use of insulators.

In one embodiment, the powder component comprises a polymethyl methacrylate polymer and the liquid component comprises methyl methacrylate.

### DESCRIPTION OF THE FIGURES

FIG. 1 shows the release forces, N as a function of the IPA myristate mass fraction. The test specimens were prepared according to Table 1.

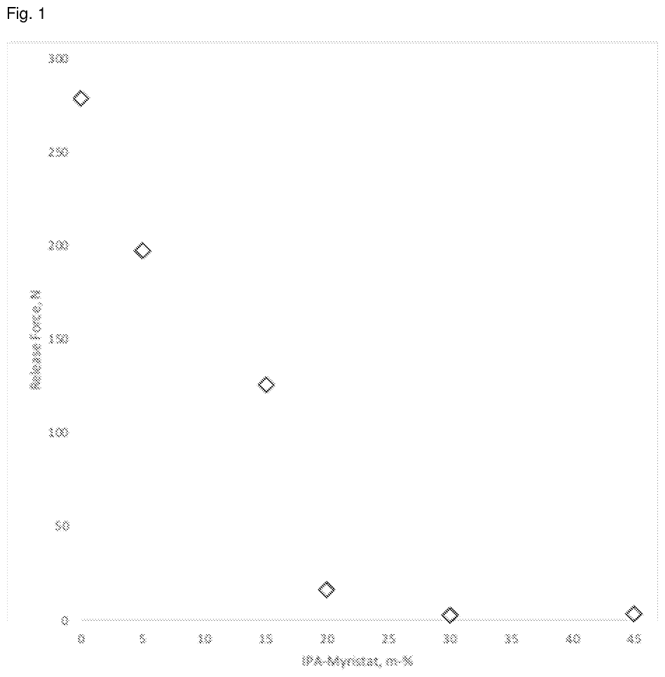

FIG. 2 shows the release forces, N as a function of the mass fraction of IPA palmitate. The test specimens were produced according to Table 2.

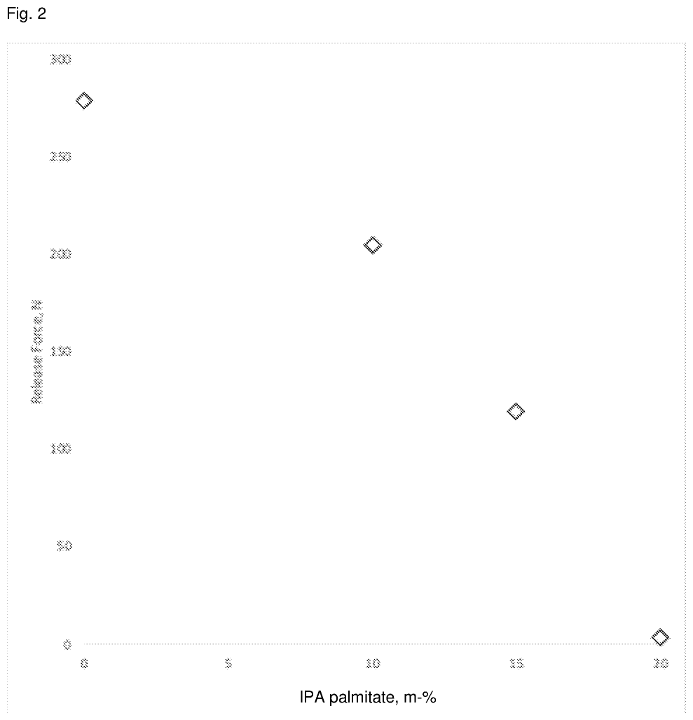

FIG. 3 shows the release forces, N (ISO 22112) of the formulation 5 according to the invention in comparison with E-SepFree, Envisiontec and “standard” model materials.

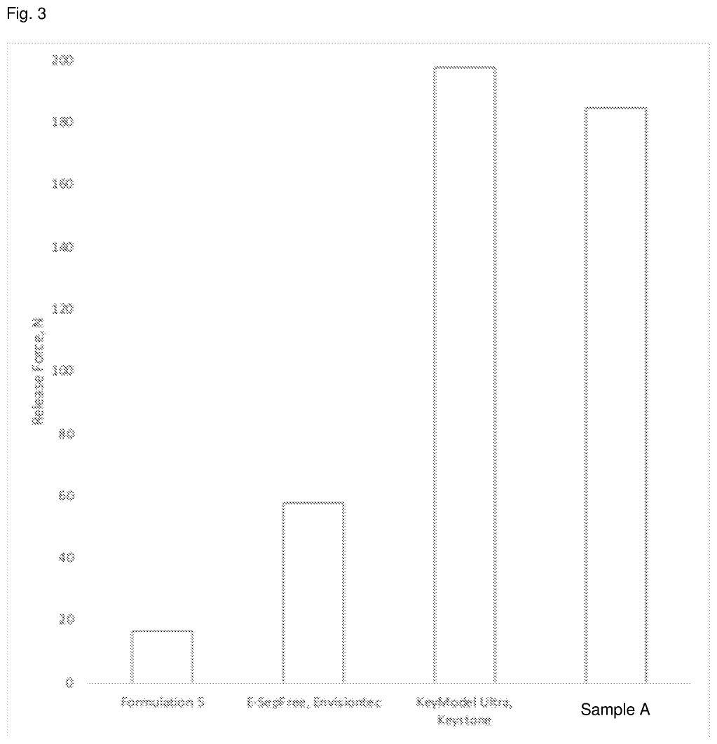

FIG. 4 shows the flexural stress (ISO 178) of the formulation 5 according to the invention in comparison with E-SepFree, Envisiontec and “standard” model materials.

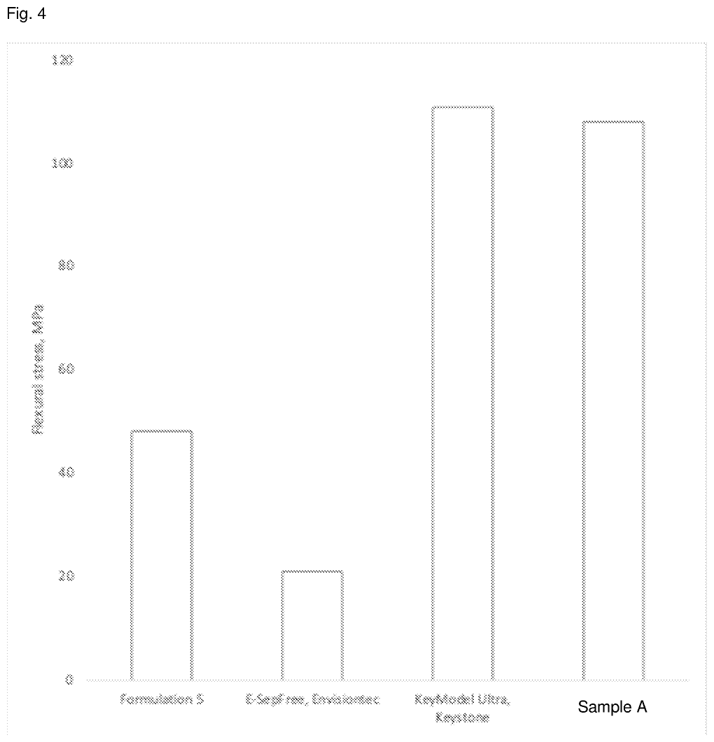

FIG. 5 shows the flexural modulus (ISO 178) of the formulation 5 according to the invention in comparison with E-SepFree, Envisiontec and “standard” model materials.

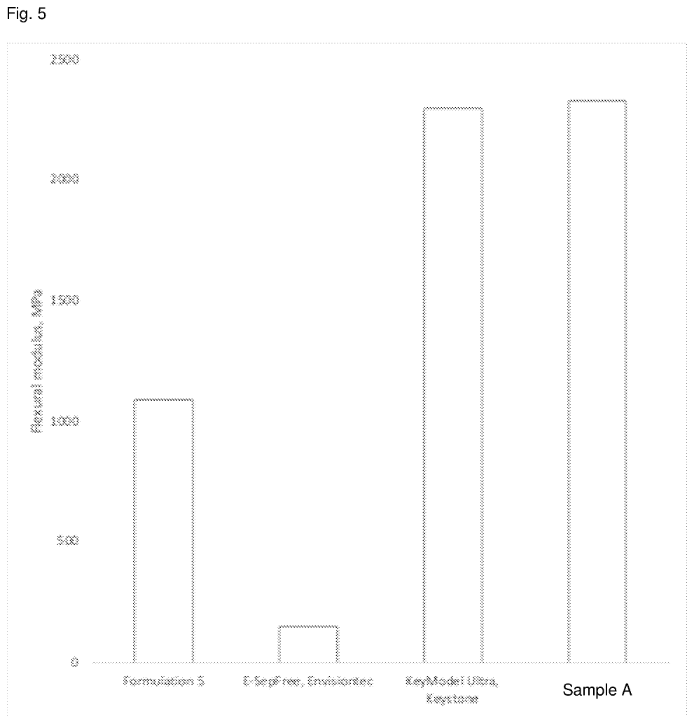

FIG. 6 shows the viscosity, mPas (DIN 53019-1) of the formulation 5 according to the invention in comparison with E-SepFree, Envisiontec and “standard” model materials.

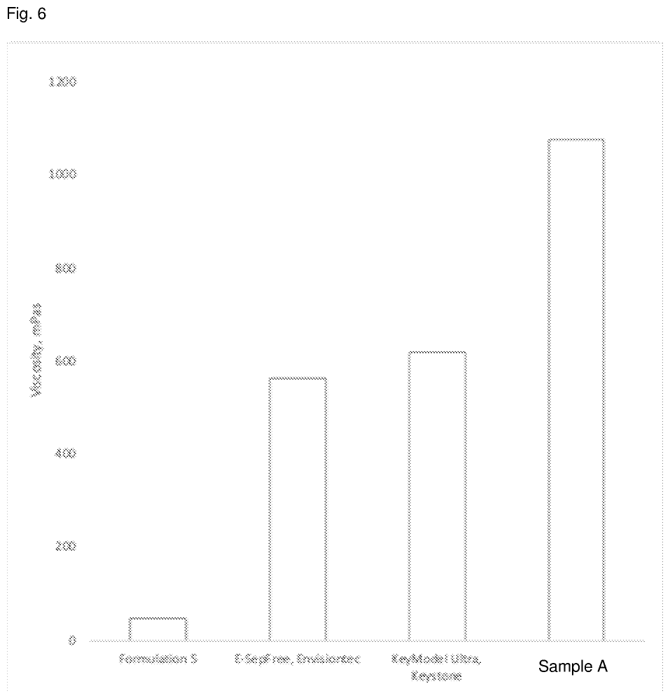

### EXAMPLES

### Preparation of Sample A

Sample A was prepared by mixing a methacrylate resin with isopropyl myristate (30 wt %). The methacrylate resin consisted of tetraethylene glycol dimethacrylate (60 wt %) and a difunctional aliphatic methacrylate (MIRAMER PU2421NT, 40 wt %). TPO (CAS No. 75980-60-8) (1.4 wt %), Irgacure 819 (CAS No. 162881-26-7) (0.6 wt %), Tinuvin P (CAS 2440-22-4) (0.1 wt %) and Tinopal OB CO (CAS 7128-64-5) (0.01 wt %) were added to the resin based on the weight of the methacrylates. The components were mixed with an Ultraturrax (30 min, 16000 min\hat{ }-1).

The light-curing resin was processed using additive manufacturing equipment (DLP-SL 3D printer: ASIGA MAXUV).

### Quantification of Liability

To compare the adhesion of a model plastic to different resins, the maximum tensile force was determined. For this purpose, a two-part specimen was produced using additive manufacturing (ASIGA MAXUV). The two parts (part 1: 10×10×5 mm, part 2: 10×20×5 mm) were bonded orthogonally to each other, with the upper side of part 1 (10×10 mm) being joined to the short side of part 2 (10×5 mm) in an L-shape. The test specimens were bonded together at a gap distance of 2 mm using a powder-liquid system (scattering plastic based on a polymethyl methacrylate polymer powder and monomeric methyl methacrylate) for the manufacture of orthodontic appliances (PL-1, pro3dure medical GmbH). The pull-off forces were measured in a device according to ISO 22112. The preload was 0.5 N, the crosshead speed 5 mm/min. The maximum force was recorded as the result.

### Mechanical Testing

The mechanical test was carried out in accordance with ISO178. Five specimens (2×2×25 mm) were tested. The crosshead speed was 10 mm/min. The results were calculated in accordance with ISO178.

TABLE 1
Formulations with myristic acid isopropyl ester

| | | Formu- | Formu- | Formu- | Formu- | Formu- | Formu- | Formu- |
|---|---|---|---|---|---|---|---|---|
| No. | IUPAC name | lation 1 | lation 2 | lation 3 | lation 4 | lation 5 | lation 6 | lation 7 |
| 1 | 2-[2-[2-[2-[2-methylprop-2- | 60 | 57 | 54 | 51 | 48 | 42 | 33 |
| | enoyloxy]ethoxy]ethoxy]ethoxy]ethyl | | | | | | | |
| | 2-methylprop-2-enoate | | | | | | | |
| 2 | 2-[[3,5,5-trimethyl-6-[2-[2-methylprop-2- | 38 | 36.1 | 34.2 | 32.3 | 30.4 | 26.6 | 20.9 |
| | enoyloxy]ethoxycarbonylamino]hexyl]carbanoyloxy]ethyl | | | | | | | |
| | 2-methylprop-2-enoate | | | | | | | |
| 3 | [phenyl-(2,4,6-trimethylbenzoyl)phosphoryl]- | 1 | 0.95 | 0.9 | 0.85 | 0.8 | 0.7 | 0.55 |
| | [2,4,6-trimethylphenyl]methanone | | | | | | | |
| 4 | diphenylphosphoryl-(2,4,6- | 1 | 0.95 | 0.9 | 0.85 | 0.8 | 0.7 | 0.55 |
| | trimethylphenyl)methanone | | | | | | | |
| 5 | propan-2-yl tetradecanoate | 0 | 5 | 10 | 15 | 20 | 30 | 45 |
| | | 100 | 100 | 100 | 100 | 100 | 100 | 100 |

TABLE 2
Formulations with palmitic acid isopropyl ester

| | | Formu- | Formu- | Formu- | Formu- |
|---|---|---|---|---|---|
| | | lation | lation | lation | lation |
| No. | IUPAC name | 8, % | 9, % | 10, % | 11, % |
| 1 | 2-[2-[2-[2-[2-methylprop-2- | 60 | 57 | 54 | 48 |
| | enoyloxy]ethoxy]ethoxy]ethoxy]ethyl | | | | |
| | 2-methylprop-2-enoate | | | | |
| 2 | 2-[[3,5,5-trimethyl-6-[2-[2-methylprop-2- | 38 | 36.1 | 34.2 | 30.4 |
| | enoyloxy]ethoxycarbonylamino]hexyl]carbanoyloxy]ethyl | | | | |
| | 2-methylprop-2-enoate | | | | |
| 3 | [phenyl-(2,4,6-trimethylbenzoyl)phosphoryl]- | 1 | 0.95 | 0.9 | 0.8 |
| | [2,4,6-trimethylphenyl]methanone | | | | |
| 4 | diphenylphosphoryl-(2,4,6- | 1 | 0.95 | 0.9 | 0.8 |
| | trimethylphenyl)methanone | | | | |
| 5 | propan-2-yl hexadecanoate | 0 | 5 | 10 | 20 |
| | | 100 | 100 | 100 | 100 |

1. A 3D printing resin comprising
a monomer or oligomer which has at least one acrylate or methacrylate subunit,
an initiator,
characterized by the fact that
the 3D printing resin comprises a saturated fatty acid or a carboxylic acid ester.

2. The 3D printing resin according to claim 1, wherein the fatty acid is an acid according to formula (1),
R¹⁰—COOH (1),
wherein R¹⁰ is a C₁₋₁₉-alkyl, in particular a C₃₋₁₉-alkyl, further in particular a C₁₁₋₁₇-alkyl.

3. The 3D printing resin according to claim 1, wherein the carboxylic acid ester is selected from an ester according to formula (2), an ester according to formula (3) or an ester prepared from a dimer fatty acid and two alcohols, in particular C₁₋₁₂-alkyl-OH,
R¹¹—C(═O)—O—R¹² (2),
R¹³—O—C(═O)—R¹⁴—C(═O)—O—R¹⁵ (3),
whereby
R¹¹ is a C₁₋₂₃-alkyl or a C₂₋₂₃-alkenyl, in particular a C₃₋₂₃-alkyl or a C₃₋₂₃-alkenyl, wherein the alkyl or the alkenyl is unsubstituted or substituted by one or more, in particular one, —OH,
R¹², R¹³ and R¹⁵ is a C₁₋₁₂-alkyl
R¹⁴ is a C₁₋₁₂-alkyl or a C₂₋₁₂-alkenyl.

4. The 3D printing resin according to claim 1, wherein the sum of all C atoms of the carboxylic acid ester is ≤20, in particular 4-20, further in particular 12-18.

5. The 3D printing resin according to claim 1, wherein the proportion by weight of the fatty acid or carboxylic acid ester relative to the total mass of the 3D printing resin is ≥5%.

6. The 3D printing resin according to claim 1, wherein the monomer or oligomer comprises, in addition to the at least one acrylate or methacrylate subunit, one or more subunits selected from an aliphatic, a cycloaliphatic, a heterocyclic, an aromatic and a urethane subunit.

7. The 3D printing resin according to claim 1, wherein the monomer is selected from a compound of formula (4), 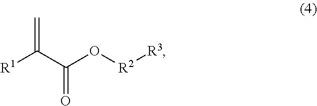
whereby
R¹ is equal to —H or —CH₃,
R² is selected from
C₁- to C₁₀-alkyl,
—[(CH₂)ₘ—O—]ₙ—(CH₂)ᵣ—,
—[(C₁₋₁₂-alkyl)ₛ-Y]ₓ—(CH₂)ₜ— 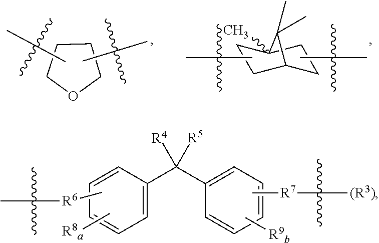
whereby
R⁴ and R⁵ are independently selected from H, —C₁₋₄-alkyl, —CF₃, phenyl, or
R⁴ and R⁵ form a ring structure comprising a hydrocarbon chain comprising 4 to 8 carbon atoms,
R⁶ is selected from —[(CH₂)ₚ—O—]_{q}—
R⁷ is selected from —O—[(CH₂)ᵥ—O—]_{w}—,
R⁸ₐ and R⁹_{b} are independently selected from —C₁₋₄-alkyl, phenyl,
Y is selected from —O—C(═O)—NR²⁰— and —NR²⁰—C(═O)—O—,
R²⁰ is a C₁₋₄-alkyl or H,
m, p, v is between 1 and 4, in particular 1 or 2,
n, q, w is between 1 and 4,
r, t, x is between 1 and 4, in particular 1 or 2,
s is between 1 and 10,
a is 0 or 1,
b is 0 or 1, and
R³ is —H, acrylate or methacrylate.

8. The 3D printing resin according to claim 1, wherein the oligomer is formed from monomers.

9. The 3D printing resin according to claim 1, wherein the initiator is a photoinitiator, in particular a photoinitiator selected from
a compound of the benzoins and benzoin ethers, in particular benzoin, benzoin methyl ether, benzoin ethyl ether and benzoin isopropyl ether, benzoin phenyl ether and benzoin acetate,
a compound of acetophenones, in particular acetophenone, 2,2-dimethoxyacetophenone, and 1,1-dichloroacetophenone,
a compound of the benzils and benzil ketals, in particular benzil, benzildimethyl ketal and benzildiethyl ketal,
a compound of the anthraquinones, in particular 2-methylanthraquinone, 2-ethylanthraquinone, 2-tert-butylanthraquinone, 1-chloroanthraquinone and 2-amylanthraquinone,
a compound of triphenylphosphines and benzoylphosphine oxides, in particular triphenylphosphines, 2,4,6-trimethylbenzoyldiphenylphosphine oxide (luzirine TPO) and bis(2,4,6-trimethylbenzoylphenyl)-phosphine oxide,
a compound of the benzophenones, in particular benzophenone and 4,4′-bis-(N,N′-dimethylamino)-benzophenone,
a compound of thioxanthones and xanthones, in particular thioxanton and xanthone,
an acridine derivative, phenazine derivative, quinoxaline derivative
1-Phenyl-1,2-propanedione-2-O-benzoyloxime,
a compound of the 1-aminophenylketones or 1-hydroxyphenylketones, in particular 1-hydroxycyclohexylphenylketone, phenyl-(1-hydroxyisopropyl)-ketone and 4-isopropylphenyl-(1-hydroxyisopropyl)-ketone.

10. The 3D printing resin according to claim 1, wherein the 3D printing resin comprises one or more additives selected from an inhibitor, a stabilizer, a filler, a colorant, a UV blocker (additive to control the depth of cure).

11. A polymer, in particular prepared using the 3D printing resin according to claim 1, comprising
Polymerized monomers and/or oligomers, and
a saturated fatty acid or a carboxylic acid ester.

12. A green compact, in particular produced using the 3D printing resin according to claim 1, comprising
Polymerized monomers and/or oligomers, wherein 60% to 80% of the double bonds of the acrylate and/or methacrylate subunits have been reacted, and
a saturated fatty acid or a carboxylic acid ester.

13. A kit-of-parts system comprising
a 3D printing resin according to claim 1 for producing a polymer having a separation effect, and
a powder component and a liquid component for the production of a plastic, wherein
the powder component comprises a polymer powder, in particular a polymer powder comprising at least one acrylate or methacrylate subunit, and
the liquid component comprises a monomer, in particular a monomer comprising at least one acrylate or methacrylate subunit.

14. A method for producing a molded part with a separation effect, in particular a dental model with a separation effect, wherein the 3D printing resin according to claim 1 is used and cured, in particular using a 3D printer.

15. A method of manufacturing a molded part, wherein
in a first step, the method according to claim 14 is carried out to obtain a first molded part, and
in a second step, the first molded part is used to obtain a second molded part which is at least partially complementary to the first molded part, the second molded part being made from a plastic, in particular using a powder component and a liquid component, wherein
the powder component comprises a polymer powder, in particular a polymer powder comprising at least one acrylate or methacrylate subunit, and
the liquid component comprises a monomer, in particular a monomer comprising at least one acrylate or methacrylate subunit, or
in a first step, a first molded part is produced from a plastic, in particular using a powder component and a liquid component, wherein
the powder component comprises a polymer powder, in particular a polymer powder comprising at least one acrylate or methacrylate subunit, and
the liquid component comprises a monomer, in particular a monomer comprising at least one acrylate or methacrylate subunit,
in a second step, the first molded part is used to obtain a second molded part which is at least sectionally complementary to the first molded part, wherein the second molded part is produced using the 3D printing resin.
